(19) United States
(12) Patent Application Publication
(10) Pub. No.: US 2007/0141690 A1
(43) Pub. Date: Jun. 21, 2007

(54) MUTANT SACCHAROMYCES CEREVISIAE STRAIN UTILIZING XYLOSE FOR ETHANOL PRODUCTION

(75) Inventors: Kaisa Karhumaa, Lund (SE); Marie-Francoise Gorwa-Grauslund, Malmo (SE)

Correspondence Address: GAUTHIER & CONNORS, LLP, 225 FRANKLIN STREET, SUITE 2300, BOSTON, MA, 02110, US

(73) Assignee: FORSKARPATENT I SYD AB, Lund (SE)

(21) Appl. No.: 11/557,363

(22) Filed: Nov. 7, 2006

Related U.S. Application Data
(63) Continuation of application No. PCT/SE05/00660, filed on May 4, 2005.

(30) Foreign Application Priority Data
May 7, 2004 (SE) 0401191-2

(51) Int. Cl. C12P 7/06; C12N 1/18

(52) U.S. Cl. 435/161; 435/254.21

(57) ABSTRACT

The present invention relates to a new xylose utilizing Saccharomyces cerevisiae strain being able to utilize xylose for ethanol production which strain is up-regulated with regard to the genes for xylose reductase (XR) and xylulose dehydrogenase (XDH) as well as xululokinase (XK) and over-expressing the non-oxidative pentose phosphate pathway (PPP) and comprising a deletion of the gene GRE3 deletion, as well as the strain has been adapted to xylose feeding.

### PRIORITY INFORMATION

This application is a continuation of International Application Serial No. PCT/SE2005/000660 filed on May 4, 2005 which claims priority to Swedish Application No. 0401191-2 filed May 7, 2004 both of which are incorporated herein by reference in their entireties.

### DESCRIPTION

Technical field

The present invention relates to a new Saccharomyces cerevisiae strain having improved properties of fermenting xylose to ethanol.

### BACKGROUND OF THE INVENTION

Ethanol is efficiently produced from hexoses by Saccharomyces cerevisiae, but recombinant S. cerevisiae strains capable of xylose utilisation are needed to expand the substrate range to lignocellulosic hydrolysate. By integration of the Pichia stipitis XYL1 and XYL2 genes encoding xylose reductase (XR) and xylitol dehydrogenase (XDH) and the endogenous XKS1 gene encoding xylulokinase (XK), stable xylose fermenting S. cerevisiae strains have been obtained. However, xylitol is secreted as a result of the difference in cofactor usage between the NAD(P)H-dependent XR and the strictly NAD⁺-dependent XDH steps.

Because of its dual cofactor utilization, XR from P. stipitis has been preferred for the construction of xylose-fermenting recombinant S. cerevisiae strains. Presently no known XR can reduce xylose to xylitol with NADH as sole cofactor. Among natural xylose-utilising yeasts Candida utilis XR exclusively uses NADPH, whereas C. shehatae, P. segobiensis, P. stipitis and Pachysolen tannophilus XRs use both NADPH and NADH in the reduction of xylose to xylitol. The C. parapsilosis XR also uses both NADPH and NADH for xylose reduction, but unlike the other yeasts it prefers NADH. Only yeasts harbouring a NADH-linked xylose reductase activity display significant alcoholic fermentation.

The ethanol yield in recombinant xylose-fermenting S. cerevisiae strains is far from the theoretical maximum of 0.51 g g⁻¹, partly because a significant fraction of the consumed xylose is secreted as xylitol. Xylitol formation in P. tannophilus has been reduced by addition of hydrogen acceptors. These compounds reoxidized NAD⁺, which is needed in the XDH reaction. Xylitol formation in recombinant S. cerevisiae has been reduced by the addition of acetoin, furfural and acetaldehyde. Xylitol formation can also be decreased by shifting the cofactor usage in the XR-step from NADPH to NADH. This was achieved by changing the intracellular pool of NADPH by blocking or reducing the oxidative pentose phosphate pathway (PPP) flux through modification of the glucose 6-phosphate dehydrogenase activity. This resulted in improved ethanol yield at the expense of impaired growth rate on glucose and decreased xylose consumption rate.

The XYL1 gene has been subjected to site-specific mutagenesis to reduce the XR-affinity for NADPH. Chemical modification studies on XR showed that cysteine and histidine residues might be involved in NADPH binding. However, mutation of three cysteine residues (Cys19, Cys27 and Cys130) to serine enhanced the apparent Km for NADPH less than 4-fold, indicating that none of these cysteine residues were directly involved in NADPH binding. A 17 times lower affinity for NADPH was achieved when the XYL1 gene carried the K270M (lysine ->methionine) mutation, whereas the affinity for NADH remained unchanged. The only difference between NADPH and NADH is the presence of a phosphate group in NADPH, and it was suggested that Lys270 binds to the phosphate group of NADPH.

Attempts have also been made to change the cofactor specificity of XDH towards NADP⁺. A NADP³⁰ recognition sequence from Thermoanaerobium brockii was introduced in the XYL2 gene resulting in equal apparent Km values for NAD⁺ and NADP⁺. However, this was achieved at the expense of reduced NAD⁺ specificity combined with unaltered NADP⁺ specificity. The mutated XYL2 gene mediated xylose growth when co expressed with the XYL1 gene in S. cerevisiae while ethanolic fermentation of xylose was not reported.

Another route is to up-regulate the xylose isomerase (XI) gene and the gene for xylose reductase (XR).

### SUMMARY OF THE PRESENT INVENTION

The present invention relates to a novel Saccharomyces cerevisiae strains expressing

xylose isomerase (XI) and xylulokinase (XK) and having been mutated by adaptation to provide improved ethanol producing yields, when consuming xylose for ethanol formation.

### BRIEF DESCRIPTION OF THE DRAWING

FIG. 1 is a graph showing aerobic growth of TMB 3055 on 50 g/l xylose.

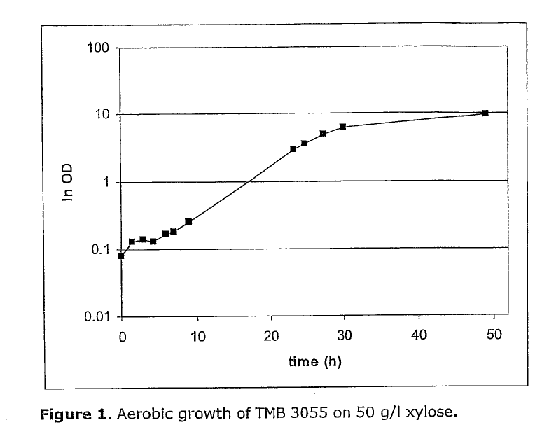

### STRAIN CONSTUCTION

Plasmid YEplacHXT-XI carrying the gene for Thermus thermophilus xylose isomerase (XI) was removed from strain TMB 3050 (DMSZ 15834) by prolonged cultivation in YPD medium. Clones lacking the plasmid were identified by replica plating on mineral medium lacking uracil. Uracil-auxotrophic clones were purified by repeated plating and one of these was named TMB 3051. TMB 3051 was transformed with plasmid pY7 (Walfridsson et al. 1997 Appl Microbiol Biotechnol 48(2):218-24), carrying the genes for xylose reductase (XR) and xylulose dehydrogenase (XDH). A control strain was made by transforming the strain TMB 3044 (precursor of TMB 3050) with pY7.

With this procedure was created a strain with all the modifications of TMB 3050 (XK and non-oxidative PPP overexpression, GRE3 deletion, adaptation) but carrying XR and XDH instead of XI.

Materials and Methods

Transformation. Standard molecular biology techniques were used {Sambrook, 1989}. The lithium acetate method was used for yeast transformation {Gietz, et al 1995}.

Cultivation conditions. Liquid cultures of S. cerevisiae were grown in YPD medium (20 g/l peptone, 10 g/l yeast extract and 20 g/l glucose) or defined mineral medium {Verduyn, 1990 #68}, supplemented with 20 g/l glucose or 50 g/l xylose as carbon source and buffered with phthalate (10.21 g/l phthalate, 2.1 g/l KOH, pH 5.5) before sterilization. The xylose used (Acros organics, New Jersey, USA) contained 0,5-1% glucose impurity equivalent to 0.5-1 g/l glucose in the medium when 50 g/l xylose was used. Plate cultures were done using YPD-agar or SC-plates (6.7 g/l Difco Yeast Nitrogen Base, 30 g/l agar). When required, uracil was added at concentration 40 μg/ml.

Pre-cultures for aerobic growth experiments with were cultivated until late exponential phase in defined mineral medium {Verduyn, 1990 #68} with 50 g/l xylose or 20 g/l glucose. The cells were washed with sterile water and inoculated at starting OD₆₂₀ of about 0.1 in in defined mineral medium {Verduyn, 1990 #68} with 50 g/l xylose. 50 ml cultures were grown in 500 ml baffled shake flasks and incubated at 30° C. with 130 rpm shaking. All cultivations were repeated at least twice. The growth rates on xylose were determined after the impurity glucose, determined by HPLC, had been consumed.

Results

Aerobic growth on xylose. TMB 3055 grew aerobically in shake-flask cultures with defined mineral medium supplied with 50 g/l xylose as the sole carbon source with growth rate of 0.16±0.01 1/h.

However, the growth of the control strain (TMB 3044), carrying the same plasmid and not having the mutation acquired by the adaptation process, was in the same range.

Oxygen Limited Fermentation,

In oxygen limited batch fermentations, xylose was consumed with rate 0.048±0.024 g xylose/g cells/h and ethanol was produced with yield 0.32±0.10. (Table 1).

TABLE 1
Oxygen limited batch fermentations with strain TMB 3055

| | q_{xylose} | Yₑₜₕₐₙₒₗ | | | |
|---|---|---|---|---|---|
| | (g xylose/g | (g/g | Y_{xylitol} | Y_{glycerol} | Y_{acetate} |
| | cells/h) | xylose) | (g/g xylose) | (g/g xylose) | (g/g xylose) |
| TMB | 0.048 ± | 0.32 ± | 0.26 ± 0.03 | 0.08 ± 0.02 | 0.03 ± 0.006 |
| 3055 | 0.024 | 0.10 | | | |

Here is the information about 3055 that I have right now.

1. A new xylose utilizing Saccharomyces cerevisiae strain being able to utilize xylose for ethanol production which strain is up-regulated with regard to the genes for xylose reductase (XR) and xylulose dehydrogenase (XDH) as well as xululokinase (XK) and over-expressing the non-oxidative pentose phosphate pathway (PPP) and comprising a deletion of the gene GRE3 deletion, as well as the strain has been adapted to xylose feeding.
